(19) United States
(12) Patent Application Publication
(10) Pub. No.: US 2007/0258888 A1
(43) Pub. Date: Nov. 8, 2007

(54) Contrast Agent for Medical Imaging Techniques and Usage Thereof

(76) Inventors: Claus Feldmann, Ettlingen (DE); Henning Braess, Lutherville (DE); Joachim Opitz, Aachen (DE); Jacqueline Merikhi, Aachen (DE)

Correspondence Address: PHILIPS INTELLECTUAL PROPERTY & STANDARDS, P.O. BOX 3001, BRIARCLIFF MANOR, NY, 10510, US

(21) Appl. No.: 10/579,155

(22) PCT Filed: Nov. 9, 2004
(86) PCT No.: PCT/IB04/52348

§ 371 Date: May 16, 2007

(30) Foreign Application Priority Data
Nov. 17, 2003 (EP) 03104236.9

(51) Int. Cl. A61K 51/12; A61B 5/055; A61B 8/13; A61K 49/04; A61K 49/06

(52) U.S. Cl. 424/1.29; 424/9.32; 424/9.41; 424/9.5

(57) ABSTRACT

A contrast agent for medical imaging techniques is described, comprising particles consisting of at least a core, the core comprising at least an oxide, mixed oxide, or hydroxide of specific elements. The particles optionally comprise shells containing or consisting of precious metal, radioactive isotopes, bio-compatibility agents, and/or antibodies. The applied imaging techniques comprise in particular magnetic resonance tomography (MRI), magnetic particle imaging, positron emission tomography (PET), single photon emission computed tomography (SPECT), computed tomography (CT), and ultrasound (US).

This invention generally pertains to the field of medicine and non-invasive imaging. The invention provides compositions and methods for imaging cells, tissues and organs in vivo and in vitro. In particular, compositions and methods are provided to enhance the imaging of cells and tissues by, e.g. positron emission tomography (PET), computed tomography (CT), magnetic resonance tomography (MRI), single photon emission computed tomography (SPECT), magnetic particle imaging, or ultrasound (US).

Contrast Agents are widely used in non-invasive imaging, in particular to diagnose cancers and abscesses. There are several types of imaging procedures conducted. In positron emission tomography (PET), two beta rays emitted from the decaying radionuclide are detected. In single photon emission computed tomography (SPECT), one beta ray emitted from the decayed radionuclide is detected. It has been found that PET provides a more exact location of the examined area, while SPECT is simpler and easier to use, and therefore used more often. Magnetic resonance imaging (MRI) is the use of a magnetic field instead of radiation to produce detailed, computer-generated pictures of organs, body areas, or the entire body. Magnetic particle imaging, a novel type of imaging technique, was invented by Philips Research, Hamburg. The basic principle is based on conventional magnetic resonance imaging (MRI). Computed tomography (CT) uses a sophisticated X-ray machine and a computer to create a detailed picture of the bodies, tissues and structures. Ultrasound (US) imaging employs ultrasonic soundwaves for generating such images.

These techniques have in common that the examination of a patient is non-invasive and free of pain. They are therefore often used for preventive medical check-up as well as for the diagnosis of different disease patterns.

For all these imaging techniques it is of major interest to enable the diagnosis of clinical pictures preferably at an early stage, with high sensitivity and high specificity. High sensitivity means that false negative diagnoses are excluded. High specificity means a reliable detection of a disease pattern, i.e. the exclusion of false positive diagnoses. Furthermore, a resolution as high as possible, preferably on cellular or molecular levels, is desirable.

Contrast agents are generally used to increase the sensitivity of the above-mentioned techniques. These contrast agents are employed to enhance the ability to distinct different areas of the examined tissue or body. Several contrast agents have been described. At present, almost exclusively ¹⁸F-marked 2-fluoro-2-deoxy-glucose (¹⁸F-FDG) is used as the commercial agent for radio diagnostics in PET-techniques. Furthermore, Gd³⁺ based metal complexes are successfully used for magnetic resonance imaging (MRI), recently. The tolerable Gd³⁺ concentration is thereby surprisingly high (several 100 mg per kg body weight). The setup of these molecular complexes is furthermore characterized by the presence of few active centers (1 to 5 atoms) in a comparably large but inactive matrix of ligands (several 100 to 1000 atoms). In the field of computed tomography (CT) hardly any contrast agents are employed at present.

However, the prior art contrast agents do not provide sufficient sensitivities with respect to the described non invasive imaging techniques. Furthermore, they are commonly limited to one specific imaging technique, respectively. Since it is desirable to verify a diagnosis with different imaging techniques, at present several agents are to be administered to a patient. Due to the low sensitivities of prior art contrast agents they are furthermore to be administered at a relatively high amount.

The aim of the present invention is to overcome the drawbacks of prior art contrast agents and to provide compositions and methods for imaging cells, tissues and organs in vivo and in vitro at a high sensitivity. Furthermore, the possibility of using different imaging techniques while employing only one single contrast agent is desired.

This aim is solved by the compositions and methods according to the independent claims of the present invention, while useful embodiments are described by the features as contained in the dependent claims.

The invention provides new imaging agents suitable for use in MRI, magnetic particle imaging, PET, SPECT, CT, and/or US techniques. These agents allow for the use of multiple imaging techniques, for example, MRI, CT and PET for diagnosis, employing a single contrast agent. It is therefore not necessary to administer different contrast agents in order to conduct an examination with different methods. Furthermore, the sensitivity of these imaging techniques using the suggested contrast agents is enhanced significantly compared to the prior art due to the large number of active centers present in or on the described agents of the invention.

In particular, the sensitivity compared to conventional Gd³⁺ based contrast agents in magnetic resonance tomography (MRI) is enhanced due to the large number of Gd³⁺ ions at the surface of the particles used as contrast agents. The same applies to positron emission tomography (PET) techniques, which are improved by the high number of ¹⁹F-ions at the surface of the particle shell if used. Moreover, a sufficient X-ray absorption is provided due to the high number of heavy atoms in the nanoscaled particles, whereby enabling imaging using computed tomography (CT). Some of the suggested agents are characterized by their magnetic characteristics, in particular by the absence of hysteresis effects as well as steep but continues course near zero field area. The latter causes a fast magnetic reversal and helps to achieve the saturation magnetisation with small external magnetic fields. This is in particular advantageous when applying magnetic resonance tomography (MRI) and magnetic particle imaging. Depending on the ingredients used, it is possible to accumulate ⁹⁹Tc atoms in the nanoscaled particles, thereby improving their sensitivity of single photon emission computed tomography (SPECT). Last but not least, the usage of precious metals imparts the suggested particles with a specific capability of reflection of ultrasound waves (US), comparable with conventionally used microscaled gas blisters.

In addition, antibodies can be immobilized on the surface of the nanoscale particles. With such a measure, a specific antibody-antigen reaction can be established, leading to specific adsorption/concentration of the contrast agent in infected tissue (e.g. cancer cells, coronar plaques). As a result, the contrast agent and the imaging process are highly specific for the respective case. Moreover, medical imaging is possible on a cellular or even molecular level.

In general, the invention provides a contrast agent for medical imaging techniques comprising particles consisting of at least a core, the core comprising at least an oxide, mixed oxide, or hydroxide of at least one element selected from the group consisting of Mg, Ca, Sr, Ba, Y, Lu, Ti, Zr, Hf, La, Ce, Pr, Nd, Sm, Eu, Gd, Tb, Dy, Ho, Er, Tm, Yb, Mo, W, Mn, Fe, Co, Ni, Cu, Zn, Cd, Si, and Bi. These particles provide an enhanced sensitivity with respect to medical imaging techniques, such as magnetic resonance tomography (MRI), magnetic particle imaging, positron emission tomography (PET), single photon emission computed tomography (SPECT), computed tomography (CT), and ultrasound (US).

In a preferred embodiment, the core of the contrast agent comprises MO, M(OH)₂, M₂O₃ or M(OH)₃ and M=Ca, Sr, Ba, Y, La, Ce, Pr, Nd, Sm, Eu, Gd, Tb, Dy, Ho, Er, Tm, Yb, Lu, or Bi, or a mixture thereof. On the one hand, these materials can serve as bearer for a shell, which is active with respect to a certain imaging technique. The usage of these materials for this purpose is advantageous, since the particle size may be adjusted accurately and simply using the manufacturing methods as described below. While the production of nanoparticles often suffers accuracy in size or amount of yield, the metal oxides according to the preferred embodiment lead to highly uniform nanoparticles at a high yield.

On the other hand, the cores consisting of oxides and hydroxides according to the preferred embodiment, may be employed as contrast agent for magnetic resonance tomography (MRI) and/or computed tomography (CT) themselves.

Preferably, the core of the contrast agent comprises Gd₂O₃, Gd(OH)₃, (Gd,M)₂O₃, (Gd,M)(OH)₃ and M=Y, La, Ce, Pr, Nd, Sm, Eu, Tb, Dy, Ho, Er, Tm, Yb, Lu or Bi, or a mixture thereof. This contrast agent is particularly useful for magnetic resonance tomography (MRI) measurements. In contrast to conventional Gd³⁺-based contrast agents for MRI, the oxide core contains a number of Gd³⁺, and potentially additional metal ions, which is by a factor of 1000 to 100000 higher but with a comparable volume. Consequently, the sensitivity of MRI can be increased significantly. Moreover, the X-ray absorption of the suggested cores is high due to the high number of heavy atoms included therein. This high number of heavy atoms (several 1000 to 100000 atoms) absorbs X-ray radiation sufficiently to allow a contrast generation with computed tomography (CT). Therefore, particles consisting of materials according to the preferred embodiment can serve as contrast agents for more than one imaging technique, for example for MRI and CT. This is in particular advantageous since different results of different techniques may be obtained from an examination of a body or a tissue, without administering different contrast agents. This is in particular useful, since the administration of different active compounds in vivo is regularly critical due to possible immunoreactions and side effects. The less the number of different agents administered and the less the amount thereof, the less the possibility that these undesired side effects or immunoreactions may appear.

Preferably, the core of the contrast agent comprises Gd₂O₃, Gd(OH)₃, (Gd,Bi)₂O₃ or (Gd,Bi)(OH)₃, or a mixture thereof. These materials are in particular advantageous since the number of Gd³⁺ ions on the particle surface (several 100 to 10000 atoms) increases the sensitivity of this core for MRI measurements significantly. In particular, the presence of Gd ions favourably affects the above described effects.

According to another preferred embodiment of the present invention, the core comprises M′M″O₄ (M′=Gd, Bi, Fe; M″=P, Nb, Ta) or M′₂M″₂O₇ (M′=Gd, Bi, Fe; M″=Si, Ti, Zr, Hf) or M′₂M″O₅ (M′=Gd, Bi, Fe; M″=Si, Ti, Zr, Hf) or M′₄(M″O₄)₃ (M′=Gd, Bi, Fe; M″=Si, Ti, Zr, Hf) or M′₂(M″O₄)₃ (M′=Gd, Bi, Fe; M″=Mo, W) or M′₂M″O₆ (M′=Gd, Bi, Fe; M″=Mo, W), or a mixture thereof.

These mixed oxides on one hand provide good processing characteristics for producing nanoparticles of a specific size and shape. On the other hand, these oxides are-suitable to be employed as contrast agent for MRI measurements, since the core contains Gd³⁺. In contrast to conventional Gd³⁺-based contrast agents for MRI, the surface of the oxide core contains a number of Gd³⁺ ions, which is by a factor of 1000 to 100000 higher but with a comparable volume. Consequently, the sensitivity of MRI can be increased significantly. Moreover, the X-ray absorption is high enough to allow a contrast generation with computed tomography (CT). As a result, a combination of MRI and CT based on only one contrast agent, allows to verify a medically diagnosis based on the specific strength of two independent methods.

Preferably, the core according to the preferred embodiment contains ⁹⁸Mo. This isotope may serve as lattice material or the lattice as doped with it. This is particularly advantageous, since ⁹⁸Mo can be transformed to ⁹⁹Tc by conventional reactor techniques. As a result, the core is also sensitive to single photon emission computed tomography (SPECT). In contrast to conventional ⁹⁹Tc-based contrast agents for SPECT, the oxide core can contain a number of ⁹⁹Tc atoms, which is by a factor of 100 to 10000 higher but with a comparable volume. Consequently, the sensitivity of SPECT can also be increased compared to contrast agents of state of the art. Nanoparticles according to this preferred embodiment may thus serve as contrast agents for three imaging techniques, namely MRI, CT and SPECT. A combination of MRI, CT and SPECT based on only one contrast agent, allows to verify a medical diagnosis based on the specific strength of three independent methods. This is in particular advantageous, since the drawback of administration of several agents is overcome by the multifunctional usability of the agent according to the preferred embodiment.

Preferably, the core is doped with ⁹⁸Mo in an amount of 0.01 mol-% to 50 mol-% Mo. This amount is specifically useful for the above described applications and makes sure that the desired amount of 98 Mo and ⁹⁹Tc, respectively, is provided.

In this context, it is specifically preferred that the core comprises one of the formulations selected from the group consisting of GdPO₄:Mo (1.0 mol-%), Gd₂Si₂O₇:Mo (5.0 mol-%), or Gd₂(WO₄)₃:Mo (10 mol-%). These formulations have specific characteristics with respect to the possible imaging techniques MRI, CT and SPECT. With contrast agents according to this specific embodiment, the co-action of the sensitivities with respect to the possible imaging techniques may be utilized in a particularly advantageous manner.

In another preferred embodiment of the present invention, a core comprises at least one of the group consisting of elementary Fe, γ-Fe₂O₃, Fe₃O₄, a ferrite material with spinel-, garnet-, or magnetoplumbite-structure, or any other hexagonal ferrite structure. In contrast to conventional Gd³⁺-based contrast agents for MRI, on a comparable volume scale the iron oxide core contains a number of magnetic centers, which is by a factor by 1000 to 100000 higher. Consequently, the sensitivity of MRI can be increased significantly. Moreover, the contrast agent, as suggested in the preferred embodiment, fulfils the special requirements of the medical imaging technique of magnetic particle imaging. The contrast agent consists of a magnetic ion oxide core that is characterized by its magnetic characteristics. In particular, the absence of hysteretic effects is beneficial. Furthermore, the core provides a steep, but continuous course of magnetization around the zero-field. This results in a fast re-magnetization behavior and the achievement of saturation of magnetization with a low external magnetic field. The cores according to this preferred embodiment of the present invention are furthermore non-agglomerated, and with one magnetic domain only.

Preferably, the spinel-structure is formed of MFe₂O₄ and M=Mn, Co, Ni, Cu, Zn, or Cd, the garnet-structure is formed of M₃Fe₅O₁₂ and M=Y, La, Ce, Pr, Nd, Sm, Eu, Gd, Tb, Dy, Ho, Er, Tm, Yb, or Lu, and the magnetoplumbit-structure is formed of MFe₁₂Oₗ₉ and M=Ca, Sr, Ba, or Zn, and the hexagonal ferrite-structure is formed of Ba₂M₂Fe₁₂O₂₂ mit M=Mn, Fe, Co, Ni, Zn, or Mg, respectively. Cores of one of these compositions provide the above-mentioned advantages in a preferable manner.

It is furthermore beneficial, if the core according to this preferred embodiment is additionally doped with Mn, Co, Ni, Cu, Zn, or F. The amount of doping preferable ranges between 0.01 and 5.00 mol-%. This doping supports the usability of the cores as contrast agents for MRI and in particular for magnetic particle imaging.

According to a particularly preferred embodiment of the present invention, the contrast agent further comprises at least one optional shell around the particle core. By introducing a shell, different advantageous effects can be reached. Firstly, additional materials can be processed in said shell, which are specifically; effective for one or more additional imaging techniques, thus leading to the possibility of an examination using more than one imaging technique. Furthermore, high compatibility can be established by choosing shell material that prevents an immune reaction of the examined body against the contrast agent particles. Moreover, a shell may be established containing biological active compounds, such as antibodies, thereby supporting a favorable distribution of the contrast agent in the examined tissue.

The at least one optional shell described can furthermore serve for the support of different imaging techniques. Preferably, at least one of the optional shells contains a radioactive isotope. This would allow for the usage of the claimed particles as a contrast agent for positron emission tomography (PET) or single photon emission computed tomography (SPECT) measurements. Thereby, it is particularly advantageous to use ¹⁹F as radioactive isotope. This leads to a high sensitivity for PET measurements.

State of the art contrast agents mainly use ¹⁸F-marked 2-fluoro-2-deoxyglucose (¹⁸F-FDG) as the commercial agent for radiodiagnostics. Characteristicly for the assembly of these molecular complexes is among others the presence of a few active centers (1 to 5 atoms) in a comparably large but inactive matrix of ligands (several 100 to 10000 atoms). Although the detection of positrons is principally possible, the high sensitivity, many radioactive decays, respectively the resulting positrons, may not be detected if the measurement terms are kept short, thereby reducing the sensitivity of the PET measurements.

The suggested radioactive isotope ¹⁹F, contained in at least one of the optional shells, overcomes this problem of the prior art by providing an enhanced sensitivity for PET measurements, since the number of active ¹⁹F ions is by the factor of 100 to 10000 higher compared to conventional contrast agents for PET. Consequently, the probability for a detection of positrons from a selected volume element is significantly increased, even if the one or other positron is absorbed due to wide-angle entrance by the detector shield.

By providing a ¹⁹F containing shell, which is effective for PET and SPECT imaging techniques, in combination with one of the above described core materials it is furthermore possible to execute further imaging techniques besides PET and SPECT, such as magnetic resonance imaging (MRI), magnetic particle imaging, computed tomography (CT) or ultrasound (US), depending on the materials processed in the core and/or any further shells. This leads to the above described advantages resulting from the possibility of applying different imaging techniques using a single contrast agent.

It is thereby particularly advantageous, that the radioactive isotope is present in an amount of 0.001 to 50 mol-%. This ensures that a sufficient amount of active centers is present in the nanoparticles.

The at least one optional shell containing the radioactive isotope has furthermore preferably a thickness of 1 to 50 nm, especially preferably between 1 and 10 nm. This thickness renders the adhesion properties feasible. Furthermore, a shell of said thickness is capable of bearing the desired number of active ¹⁹F ions.

In a more preferred embodiment of the present invention, the core further comprises at least one shell consisting of precious metal, preferably Au, Pt, Ir, Os, Ag, Pd, Rh or Ru and more preferably Au. This allows a contrast generation using ultrasound (US), thereby enabling the particles according to the preferred embodiment to be used as contrast agent for ultrasound measurements. The particles thereby provide reflection capabilities for ultrasound (US) comparable to gas microbubbles, as conventionally used.

Preferably, the at least one optional shell of precious metal is applied to a core consisting of Fe, γ-Fe₂O₃, Fe₃O₄, or a ferrite material as described above. This renders a combination of MRI and US possible, based on only one contrast agent, allowing the verification of a medical diagnosis based on the specific strength of two independent methods.

Preferably, the at least one optional shell of precious metal covers the core completely. In doing so, the shell preferably has a thickness of 1 to 50 nm, and more preferably 1 to 10 nm. This design features particularly useful reflection capabilities for ultrasound measurements.

According to another preferred embodiment of the present invention, at least one further shell is present, providing biocompatibility. This ensures, that after administering the contrast agent to a living organism, no immune reaction against this agent takes place, which allows the application in vivo. This shell particularly consists of SiO₂, a polyphosphate (e.g. calcium polyphosphate), an amino acid (e.g. asparagin acid), an organic polymer (e.g. polyethylene glycol/PEG, polyvinyl alcohol/PVA, polyamide, polyacrylate, polyurea), a biopolymer (e.g. polysaccharide, such as dextrane, xylane, glycogene, pectine, cellulose, or polypeptide, such as collagene, globuline), cysteine, or a peptide with a high amount of asparagine, or a phospholipide.

This biocompatibility shell preferably covers the core completely and has a thickness of 1 to 50 nm, preferably 10 to 50 nm. It is thereby ensured that the adhesion characteristics of said shell to the core are convenient, thereby preventing any immunoreactions.

According to a further preferred embodiment of the present invention, at least one further shell is present, containing at least one antibody. By immobilizing antibodies on the surface of the nanoscale particles, a specific antibody-antigene reaction can be established. This leads to specific adsorption/concentration of the contrast agent in infected tissue (e.g. cancer cells, coronar plaques). As a result, the contrast agent and the imaging process are highly specific to the respective case. Moreover, medical imaging is possible on a cellular or even molecular level. Dependent on the desired purpose, one or more antibodies may be employed. In the following, several examples of antibodies are given, that may be used for the described application. However, this list is not intended to be exhaustive, since other antibodies are also applicable, in particular, antibodies that are available at some future date only.

Trastuzumab (detection of breast cancer)

Rituximab (detection of Non-Hodgkin lymphome)

Alemtuzumab (detection of chronical-lymphocytic leukemia)

Gemtuzumab (detection of acute myelogene leukemia)

Edrecolomab (detection of bowel cancer)

Ibritumomab (detection of Non-Hodgkin-lymphome)

Cetuximab (detection of bowel cancer)

Tositumomab (detection of Non-Hodgkin-lymphome)

Epratuzumab (detection of Non-Hodgkin-lymphome)

Bevacizumab (detection of lung and bowel cancer)

anti-CD33 (detection of acute myelogene leukemia)

Pemtumomab (detection of ovary and stomach cancer)

Mittumomab (detection of lung and skin cancer)

anti-MUC 1 (detection of Adenocarcinoma)

anti-CEA (detection of Adenocarcinoma)

anti-CD 61 (detection of coronar deposits/plaques)

Preferably, the at least one antibody is a tumour specific antibody. This allows for the usage of the contrast agents for tumour prevention and treatment, involving the identification and localisation of specific tumours.

The at least one antibody containing shell may further contain one or more proteins, preferably the HIV-tat protein. This facilitates the passage of these agents through e.g. a cell membrane. This advantageously enables examinations involving intracellular procedures and metabolisms.

According to a preferred embodiment of the present invention, the core of the contrast agents has a spherical, oval or lens-shape. Thereby, an optimized volume to surface ratio is provided. Furthermore, the distribution of said particles in the examined tissue or body is facilitated. Preferably, the core has a diameter of 1 to 500 nm, preferably 5 to 50 nm. This comes up to the size of several proteins and bioorganic compounds as present in human and animal organisms. Thereby, these particles are easily involved in metabolism processes, as for example intercellular exchange reactions, thereby facilitating the transport and adsorption of the contrast agents at areas of interest.

The invention further provides pharmaceutical formulations comprising the contrast agent of the invention and a pharmaceutically acceptable excipient, wherein the contrast agent is formed according to any of the above described embodiments, and wherein the formulation is suitable for administration as an imaging enhancing agent and the contrast agent is present in an amount sufficient to enhance a magnetic resonance tomography (MRI) image, a magnetic particle imaging image, a positron emission tomography (PET) image, a single photon emission computed tomography (SPECT) image, a computed tomography (CT) image, or an ultrasound (US) image. These pharmaceuticals can be administered by any means in any appropriate formulation.

The formulations of the invention can include pharmaceutically acceptable carriers that can contain a physiologically acceptable compound that acts, e.g. to stabilize the composition or to increase or to decrease the absorption of the agent and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipients or other stabilizers and/or buffers. Detergents can also be used to stabilize the composition or the increase or decrease the absorption of the pharmaceutical composition. Other physiologically acceptable compounds include wetting agents, emulsifying agents, dispersing agents or preservatives that are particularly useful for preventing the growth or action of microorganisms. Various preservatives are well known, e.g. ascorbic acid. One skilled in the art would appreciate that the choice of a pharmaceutically acceptable carrier, including a physiologically acceptable compound depends, e.g. on the route of administration and on the particular physio-chemical characteristics of any co-administered agent.

In one aspect, the composition for administration comprises a contrast agent of the invention in a pharmaceutically acceptable carrier, e.g., an aqueous carrier. A variety of carriers can be used, e.g., buffered saline and the like. These solutions are sterile and generally free of undesirable matter. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected.

The invention may be applied according to a method for in vivo or in vitro imaging a cell, a tissue, an organ or a full body comprising the following steps: a) providing a pharmaceutical formulation comprising the contrast agent of the invention and a pharmaceutically acceptable excipient, wherein the contrast agent is formed according to any of the above described embodiments, and wherein the formulation is suitable for administration as an imaging enhancing agent and the contrast agent is present in an amount sufficient to enhance a magnetic resonance tomography (MRI) image, a magnetic particle imaging image, a positron emission tomography (PET) image, a single photon emission computed tomography (SPECT) image, a computed tomography (CT) image, or an ultrasound (US) image; b) providing an imaging device wherein the imaging device is a magnetic resonance tomography (MRI) device, a magnetic particle imaging device, a positron emission tomography (PET) device, a single photon emission computed tomography (SPECT) device, a computed tomography (CT) device, or an ultrasound (US) device, or equivalent; c) administering the pharmaceutical formulation in an amount sufficient to generate the cell, tissue or body image; and d) imaging the distribution of the pharmaceutical formulation of step a) with the imaging device, thereby imaging the cell, tissue or body.

The pharmaceutical formulations of the invention can be administered in a variety of unit dosage forms, depending upon the particular cell or tissue or cancer to be imaged, the general medical condition of each patient, the method of administration, and the like. Details on dosages are well described on the scientific and patent literature. The exact amount and concentration of contrast agent or pharmaceutical of the invention and the amount of formulation in a given dose, or the “effective dose” can be routinely determined by, e.g. the clinician. The “dosing regimen” will depend upon a variety of factors, e.g. whether the cell or tissue or tumour to be imaged is disseminated or local, the general state of the patient's health, age and the like. Using guidelines describing alternative dosing regimens, e.g. from the use of other imaging contrast agents, the skilled artisan can determine by routine trials optimal effective concentrations of pharmaceutical compositions of the invention.

The pharmaceutical compositions of the invention can be delivered by any means known in the art systematically (e.g. intra-venously), regionally or locally (e.g. intra- or peri-tumoral or intra-cystic injection, e.g. to image bladder cancer) by e.g. intra-arterial, intra-tumoral, intra-venous (iv), parenteral, intra-pneural cavity, topical, oral or local administration, as sub-cutaneous intra-zacheral (e.g. by aerosol) or transmucosal (e.g. voccal, bladder, vaginal, uterine, rectal, nasal, mucosal), intra-tumoral (e.g. transdermal application or local injection). For example, intra-arterial injections can be used to have a “regional effect”, e.g. to focus on a specific organ (e.g. brain, liver, spleen, lungs). For example intra-hepatic artery injection or intra-carotid artery injection. If it is decided to deliver the preparation to the brain, it can be injected into a carotid artery or an artery of the carotid system of arteries (e.g. ocipital artery, auricular artery, temporal artery, cerebral artery, maxillary artery etc.).

These and other aspects of the invention will be apparent from and elucidated with reference to the embodiments described hereinafter.

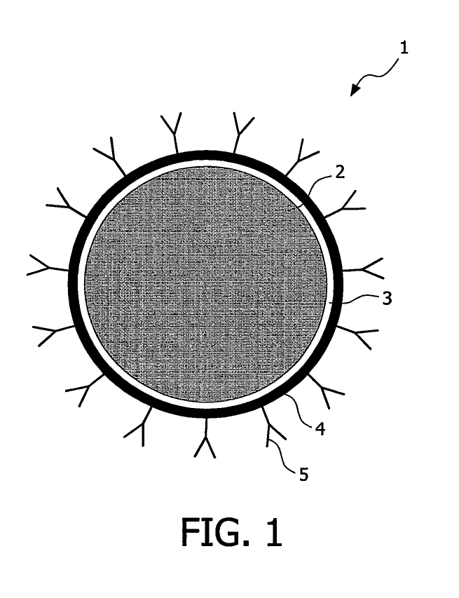

FIG. 1 is a cross-sectional view of a particle according to the present invention. This particle 1 comprises a core 2, optionally covered by shells 3 to 5.

In the following several examples are given, according to which the invention may be accomplished.

### EXAMPLE 1

0.92 g Gd(CH₃COO)₃×H₂O are suspended in 50 ml Diethylenglycole. The suspension is stirred steadily and heated to 140° C. 0.2 ml of a 1 molar caustic soda are added. In the following it is heated to 180° C. under distillation conditions for 4 hours. After cooling a suspension results, which contains nanoscaled Gd₂O₃ with a particle diameter of about 20 nm. By centrifugation, followed by suitable washing processes (e.g. repeated resuspending of the solid in ethanol and/or acetone, repeated centrifugation) the nanoscaled particles may be separated from the primary suspension and transferred to an aqueous suspension (e.g. isotonic solution or phosphate buffer). This may already be used as a contrast agent for MRI and/or CT.

Starting from the diethylenglycole based primary suspension as well as a secondary, aqueous suspension, the nanoscaled Gd₂O₃ particles may be further modified. 10 ml of an aqueous solution, containing 50 mg asparagine acid and 100 mg tetraethylorthosilicate, may be added. Thereby, a first asparagine acid containing shell of SiO₂ may be built upon the nanoparticles. The thickness of the first shell thereby amounts to approximately 15 nm. At last, 2 ml of an aqueous 10⁻⁴ molar solution of an antibody (e.g. anti-CEA) or a histidine-modified antibody (e.g. histidine-modified anti-CEA) may be added, and the antibody may be attached to the asparagine acid/SiO₂-layer by amide-bridging as a second shell. This intermediate may be used as a specific contrast agent for MRI and/or CT.

To this suspension 2.5 ml of a 0.1 molar Na¹⁹F-solution are added over 10 min. After further 10 min, the solid is centrifuged and again resuspended to an aqueous suspension (e.g. isotonic solution or phosphate buffer). An exchange of about 20 mol-% of the oxide ions with fluoride ions in the surface of the nanoscaled particles is achieved. The resulting suspension may be used as a specific contrast agent for MRI and/or CT and/or PET.

### EXAMPLE 2

1.85 g Gd(CH₃COO)₃×H₂O and 1.95 g Lu(CH₃COO)₃×H₂O are suspended in 50 ml diethylenglycole. The suspension is stirred steadily and heated to 140° C. 0.5 ml of a 1 molar caustic soda are added. In the following, it is heated to 190° C. under distillation conditions for 4 hours. After cooling a suspension results, containing nanoscaled GdLuO₃ with a particle diameter of about 45 nm. By centrifugation followed by adequate washing processes (e.g. repeated resuspending of the solid in ethanol and/or acetone, repeated centrifugation), the nanoscaled particles in the primary suspension may be separated and transferred into an aqueous suspension (e.g. isotonic solution or phosphate buffer). This may already be used as contrast agent for MRI and/or CT.

Starting from the diethylenglycole based primary suspension as well as from a secondary, aqueous suspension, the nanoscaled GdLuO₃ particles may be further modified. 20 ml of an aqueous 10⁻³ molar solution, containing asparagine acid modified dextrane, may be added. Thereby, a first dextrane containing shell may be built upon the nanoparticles. The thickness of the first shell thereby amounts to approximately 20 nm. At last, 3 ml of an aqueous 10⁻⁴ molar solution of an antibody (e.g. anti-CEA) or a histidine-modified antibody (e.g. histidine-modified anti-CEA) may be added, and the antibody may be attached to the asparagine acid/dextrane-layer by amide-bridging as a second shell. This intermediate may be used as a specific contrast agent for MRI and/or CT.

To this suspension 4 ml of a 0.1 molar H¹⁹F-solution are added over 10 min. After further 10 min, the solid is centrifuged and again resuspended to an aqueous suspension (e.g. isotonic solution or phosphate buffer). An exchange of about 20 mol-% of the oxide ions with fluoride ions in the surface of the nanoscaled particles is achieved. The resulting suspension may be used as a specific contrast agent for MRI and/or CT and/or PET.

### EXAMPLE 3

1.48 g Gd(CH₃COO)₃×H₂O and 0.35 g BiCl₃ are suspended in 50 ml diethylenglycole. The suspension is steadily stirred and heated to 140° C. 0.2 ml of a 1-molar caustic soda are added. In the following it is heated to 180° C. under distillation conditions for 4 hours. After cooling a suspension results, containing nanoscaled Gd_{1,6}Bi_{0,4}O₃ with a particle diameter of approximately 30 nm. Nanoscaled particles may be separated from the primary suspension by centrifugation followed by appropriate washing processes (e.g. repeated resuspending of the solid in ethanol and/or acetone, repeated centrifugation) and transferred into an aqueous suspension (e.g. isotonic solution or phosphate buffer). 2.5 ml of a 0.1 molar H¹⁹F solution are added over 10 min. After further 10 min the solid is centrifuged again and resuspended to an aqueous suspension (e.g. isotonic solution or phosphate buffer). An exchange of approximately 20 mol-% of the oxide ions with fluoride ions in the surface of the nanoscale particles is achieved. The resulting suspension may be used as a contrast agent for MRI and/or CT and/or PET.

### EXAMPLE 4

1.48 g Gd(CH₃COO)₃×H₂O and 12 mg MoCl₅ are suspended in 15 ml diethylenglycole. The suspension is steadily stirred and heated to 140° C. 5 ml of a solution of 0.6 g (NH₄)H₂PO₄ in water are added. In the following it is heated to 180° C. under distillation conditions for 4 hours. After cooling, a suspension results, containing nanoscaled GdPO₄:Mo (1 mol-%) with a particle diameter of approximately 20 nm. By centrifugation, followed by appropriate washing processes (e.g. repeated resuspending the solid in ethanol or acetone, repeated centrifugation), the nanoscale particles may be separated from the primary suspension and transferred into an aqueous suspension (e.g. isotonic solution or phosphate buffer). By radiating in an appropriate reactor, the desired amount of ⁹⁸Mo may be converted ⁹⁹Tc. The resulting suspension may be used as a contrast agent for MRI and/or CT and/or SPECT.

### EXAMPLE 5

0.92 g Gd(CH₃COO)₃×H₂O, 0.87 g BiCl₃ and 38 mg MoCl₅ are suspended in 50 ml diethylenglycole. The suspension is steadily stirred and heated to 140° C. 0.63 g tetraethylorthosilicate are added. In the following, it is heated to 190° C. under distillation conditions for 8 hours. After cooling a suspension results, containing nanoscaled (Gd,Bi)SiO₅:Mo (5 mol-%) with a particle diameter of approximately 35 nm. By centrifugation, followed by appropriate washing processes (e.g. repeated resuspending the solid in ethanol and/or acetone, repeated centrifugation) the nanoscaled particles may be separated from the primary suspension and transferred into an aqueous suspension (e.g. isotonic solution or phosphate buffer). By radiating with an appropriate reactor the desired amount of ⁹⁸Mo may be converted into ⁹⁹Tc. The resulting suspension may be used as contrast agent for MRI and/or CT and/or SPECT.

Starting from the diethylenglycole-based primary suspension as well as the secondary, aqueous suspension, the nanoscaled (Gd,Bi)SiO₅:Mo(⁹⁹Tc) particles may be furthermore defined. 10 ml of an aqueous solution, containing 50 mg asparagine acid and 100 mg tetraethylorthosilicate, may be added to the suspension over 1 hour, respectively. Thereby, a first asparagine acid containing shell of SiO₂ may be built on the nanoparticles. The thickness of the first shell is thereby approximately 15 nm. At last, 2 ml of an aqueous 10⁻⁴ molar solution of an antibody (e.g. anti-CEA) or an histidine-modified antibody (e.g. histidine-modified anti-CEA) may be added and the antibody may be bonded to the asparagine acid/SiO₂-layer by amide-bridging. The resulting suspension may be used as a contrast agent for MRI and/or CT and/or SPECT.

### EXAMPLE 6

1.85 g Gd(CH₃COO)₃×H₂O, 2.56 g WOCl₄ and 0.21 g MoOCl₄ are suspended in 50 ml diethylenglycole. The suspension is steadily stirred and heated to 190° C. under distillation conditions for 4 hours. After cooling a suspension results, containing nanoscaled Gd₂(WO₄)₃:Mo (10 mol-%) with a particle diameter of approximately 30 nm. By centrifugation, followed by appropriate washing processes (e.g. repeated resuspending the solid in ethanol and/or acetone, repeated centrifugation) the nanoscaled particles may be separated from the primary suspension and transferred into an aqueous suspension (e.g. isotonic solution or phosphate buffer).

Starting from the diethylenglycole-based primary suspension as well as the secondary, aqueous suspension, the nanoscaled Gd₂(WO₄)₃:Mo particles may be further modified. 20 ml of an aqueous 10⁻³ molar solution with asparagine acide modified dextrane may be added. Thereby, a first shell of dextrane may be built on the nanoparticles, having a thickness of approximately 20 nm. Finally, 3 ml of an aqueous 10⁻⁴ molar solution of an antibody (e.g. anti-CEA) or an histidine-modified antibody (e.g. histidine-modified anti-CEA) may be added and the antibody may be bonded to the asparagine acid/dextrane-layer by amide-bridging.

By radiating in an appropriate reactor the desired amount of ⁹⁸Mo may be converted into ⁹⁹Tc. The resulting suspension may be used as a contrast agent for MRI and/or CT and/or SPECT.

### EXAMPLE 7

5 g Fe(CH₃COO)₂ and 125 mg Fe(C₂O₄)×2H₂O are suspended in 50 ml diethylenglycole. The suspension is steadily stirred in a reduction gas atmosphere (N₂:H₂=95:5) and heated to 140° C. 0.5 ml of an 1 molar caustic soda solution are added. In the following, it is heated to 180° C. in reduction gas for 2 hours. After cooling, a suspension results, containing nanoscaled Fe₃O₄ with a particle diameter of approximately 15 nm. After cooling, the iron oxide particles may be separated from their primary suspension by centrifugation, followed by appropriate washing processes (e.g. repeated resuspending the solid in ethanol and/or acetone, repeated centrifugation) and transferred into an aqueous suspension (e.g. isotonic solution or phosphate buffer). This may already be used as a contrast agent for MRI and/or magnetic particle imaging.

Starting from the diethylenglycole-based primary suspension as well as the secondary, aqueous suspension, the nanoscaled Fe₃O₄ particles may be further modified. 10 ml of an aqueous solution containing 100 mg asparagine acide and 500 mg tetraethylorthosilicate, may be added to the suspension over 1 hour, respectively. Thereby, an asparagine acid containing shell of SiO₂ may be built on the nanoparticles. The thickness of the shell is thereby approximately 15 nm. Finally, 2 ml of an aqueous 10⁻⁴ molar solution of an antibody (e.g. bevacizumab) or an histidine-modified antibody (e.g. histidine-modified bevacizumab) may be added and the antibody may be bonded to the asparagine acid/SiO₂-layer by amide-bridging. This product may be used as a specific contrast agent for MRI and/or magnetic particle imaging.

### EXAMPLE 8

10 g Fe(CH₃COO)₂ and 250 mg Fe(C₂O₄)×2H₂O are suspended in 50 ml diethylenglycole. The suspension is steadily stirred and heated to 140° C. 1.0 ml of a 1 molar caustic soda solution are added. In the following, it is heated to 180° C. for 2 hours. A suspension is achieved, containing nanoscaled γ-Fe₂O₃ with a particle diameter of approximately 35 nm. To this suspension a solution of 420 mg NaAuCl₄ x 2H₂O in water is added by 180° C. over 1 hour. Thereby, a homogenous coverage of the iron oxide surfaces with elementary gold with a thickness of approximately 5 nm may be achieved. After cooling, the gold covered iron oxide particles may be separated from the primary suspension by centrifugation, followed by appropriate washing processes (e.g. repeated resuspending the solid in ethanol and/or acetone, repeated centrifugation) and transferred into an aqueous suspension (e.g. isotonic solution or phosphate buffer). This can already be used as a contrast agent for MRI and/or magnetic particle imaging and/or US.

Starting from the diethylenglycole-based primary suspension as well as the secondary, aqueous suspension, the gold-covered nanoscaled iron oxide particles may be further modified. 10 ml of an aqueous solution with 50 mg cysteine and 100 mg tetraethylorthosilicate may be added to the suspension, respectively. Thereby, a second cysteine containing shell of SiO₂ may be built upon the gold layer. The thickness of the second layer is approximately 10 nm. Finally, 2 ml of an aqueous 10⁻⁴ molar solution of an antibody (e.g. anti-CEA) or an histidine-modified antibody (e.g. histidine-modified anti-CEA) may be added and the antibody may be bonded to the cysteine/SiO₂-layer by amide-bridging. This product may be used as a specific contrast agent for MRI and/or magnetic particle imaging and/or US.

### EXAMPLE 9

20 g Fe(CH₃COO)₂ and 450 mg Fe(C₂O₄)×2H₂O are suspended in 50 ml diethylenglycole. The suspension is steadily stirred and heated to 140° C. 2 ml of a 1 molar caustic soda solution are added. In the following, it is heated to 180° C. for 3 hours. A suspension is achieved, containing nanoscaled γ-Fe₂O₃ with a particle diameter of approximately 50 nm. After cooling, the iron oxide particles may be separated from the primary suspension by centrifugation, followed by appropriate washing processes (e.g. repeated resuspending the solid in ethanol and/or acetone, repeated centrifugation) and transferred into an aqueous suspension (e.g. isotonic solution or phosphate buffer). This may already be used as a contrast agent for MRI and/or magnetic particle imaging.

Starting from the diethylenglycole-based primary suspension as well as a secondary, aqueous suspension, the nanoscaled γ-Fe₂O₃ particles may be further modified. 20 ml of an aqueous 10⁻³ molar solution with dextrane, may be added to the suspension, respectively. Thereby, a shell of dextrane may be built upon the nanoparticles, having a thickness of approximately 20 nm. This product may be used as a specific contrast agent for MRI and/or magnetic particle imaging.

### EXAMPLE 10

5 g Fe(CH₃COO)₂ and 25 mg Fe(C₂O₄)×2H₂O are suspended in 50 ml diethylenglycole. The suspension is steadily stirred in a reduction gas atmosphere (N₂:H₂=95:5) and heated to 140° C. 0.2 ml of a 1 molar caustic soda solution are added. In the following, it is heated to 180° C. for 2 hours under reduction gas. After cooling, a suspension results, containing nanoscaled Fe₃O₄ with a particle diameter of approximately 20 nm. A solution of 680 mg NaAuCl₄×2H₂O in water is added to this suspension by room temperature over 1 hour. Thereby, a homogenous coverage of the iron oxide surface with elementary gold with a layer thickness of approximately 8 mn is achieved. The gold covered iron oxide particles may be separated from the primary suspension by centrifugation, followed by appropriate washing processes (e.g. repeated resuspending the solid in ethanol and/or acetone, repeated centrifugation) and transferred into an aqueous suspension (e.g. isotonic solution or phosphate buffer). This may already be used as a contrast agent for MRI and/or magnetic particle imaging and/or US.

Starting from the diethylenglycole-based primary suspension as well as the secondary, aqueous suspension, the gold covered, nanoscaled iron oxide particles may be further modified. 10 ml of an aqueous 10⁻³ molar solution with cysteine-modified dextrane, may be added to the suspensions, respectively. Thereby, a second shell of dextrane may be built upon the gold layer by establishing AuS-bridges, the second shell having a thickness of approximately 15 nm. Finally, 10 ml of an aqueous 10⁻⁴ molar solution of an antibody (e.g. anti-CEA) or an histidine-modified antibody (e.g. histidine-modified anti-CEA) may be added and the antibody may be bonded to the cysteine-dextrane layer by amide-bridging. This product may be used as a specific contrast agent for MRI and/or magnetic particle imaging and/or US.

### EXAMPLE 11

10 g Fe(CH₃COO)₂ and 150 mg Fe(C₂O₄)×2H₂O are suspended in 50 ml diethylenglycole. The suspension is steadily stirred and heated to 140° C. 0.2 ml of a 1 molar caustic soda solution are added. In the following, it is heated to 180° C. for 2 hours. A suspension is achieved, containing nanoscaled γ-Fe₂O₃ with a particle diameter of approximately 35 nm. A solution of 420 mg NaAuCl₄×2H₂O in water is added to this suspension at 180° C. over 1 hour. Thereby, a homogenous coverage of the iron oxide surfaces with elementary gold in a layer thickness of approximately 5 nm is achieved. After cooling, the gold covered iron oxide particles may be separated from the primary suspension by centrifugation, followed by appropriate washing processes (e.g. repeated resuspending the solid in ethanol and/or acetone, repeated centrifugation) and transferred into an aqueous suspension (e.g. isotonic solution or phosphate buffer). This may then be used as a contrast agent for MRI and/or magnetic particle imaging and/or US.

Starting from the diethylenglycole-based primary suspension as well as the secondary, aqueous suspension, the gold covered, nanoscaled iron oxide particles may be further modified. 10 ml of an aqueous solution with 50 mg cysteine and 100 mg tetraethylorthosilicate, may be added to the suspensions, respectively. Thereby, a second cysteine-containing shell of SiO₂ may be established on the gold layer. The thickness of the second layer is approximately 10 nm. Finally, 2 ml of an aqueous 10⁻⁴ molar solution of an antibody (e.g. anti-CEA) or an histidine-modified antibody (e.g. histidine-modified anti-CEA) may be added and the antibody may be bonded to the cysteine/SiO₂-layer by amide-bridging. This product may be used as a specific contrast agent for MRI and/or magnetic particle imaging and/or US.

The invention has been described herein with reference to certain preferred embodiments. However, as obvious variations thereon will become apparent to those skilled in the art, the invention is not to be considered as limited thereto. In particular, other combinations and preparations of metal oxides than described in one of the examples may serve as contrast agents according to the present invention. Furthermore, the given examples of antibodies that may be used according to the present invention are not intended to be exhaustive, since other antibodies are also applicable, in particular, antibodies that are available at some future date only. Any reference signs in the claims do not limit the scope of the invention. The term “comprising” is to be understood as not excluding other elements or steps and the term “a” or “an” does not exclude a plurality.

1. A contrast agent for medical imaging techniques, comprising particles (1) consisting of at least a core (2), the core (2) comprising at least an oxide, mixed oxide, or hydroxide of at least one element selected from the group consisting of Mg, Ca, Sr, Ba, Y, Lu, Ti, Zr, Hf, La, Ce, Pr, Nd, Sm, Eu, Gd, Tb, Dy, Ho, Er, Tm, Yb, Mo, W, Mn, Fe, Co, Ni, Cu, Zn, Cd, Si, and Bi.

2. The contrast agent according to claim 1, wherein the core (2) comprises MO, M(OH)₂, M₂O₃ or M(OH)₃ and M=Ca, Sr, Ba, Y, La, Ce, Pr, Nd, Sm, Eu, Gd, Tb, Dy, Ho, Er, Tm, Yb, Lu, or Bi, or a mixture thereof.

3. The contrast agent according to claim 1, wherein the core (2) comprises Gd₂O₃, Gd(OH)₃, (Gd,M)₂O₃, (Gd,M)(OH)₃ and M=Y, La, Ce, Pr, Nd, Sm, Eu, Tb, Dy, Ho, Er, Tm, Yb, Lu or Bi, or a mixture thereof.

4. The contrast agent according to claim 1, wherein the core (2) comprises Gd₂O₃, Gd(OH)₃, (Gd,Bi)₂O₃ or (Gd,Bi)(OH)₃, or a mixture thereof.

5. The contrast agent according to claim 1, wherein the core (2) comprises M′M″O₄(M′=Gd, Bi, Fe; M″=P, Nb, Ta) or M′₂M″₂O₇(M′=Gd, Bi, Fe; M″=Si, Ti, Zr, Hf) or M′₂M″O₅(M′=Gd, Bi, Fe; M″=Si, Ti, Zr, Hf) or M′₄ (M″O₄)₃(M′=Gd, Bi, Fe; M″=Si, Ti, Zr, Hf) or M′₂(M″O₄)₃(M′=Gd, Bi, Fe; M″=Mo, W) or M′₂M″O₆(M′=Gd, Bi, Fe; M″=Mo, W), or a mixture thereof.

6. The contrast agent according to claim 5, wherein the core (2) contains ⁹⁸Mo as lattice material and/or the lattice is doped with ⁹⁸Mo.

7. The contrast agent according to claim 6, wherein the amount of doping ranges between 0.01 and 50 mol-%.

8. The contrast agent according to claim 5, wherein the core (2) comprises one of the formulations selected from the group consisting of GdPO₄:Mo (1.0 mol-%), Gd₂Si₂O₇:Mo(5.0 mol-%), or Gd₂(WO₄)₃:Mo(10 mol-%).

9. The contrast agent according to claim 1, wherein the core (2) comprises at least one of the group consisting of elementary Fe, γ-Fe₂O₃, Fe₃O₄, a ferrite material with spinel-, garnet-, or magnetoplumbite-structure, or any other hexagonal ferrite structure.

10. The contrast agent according to claim 9, wherein the spinel-structure is formed of MFe₂O₄ and M=Mn, Co, Ni, Cu, Zn, or Cd.

11. The contrast agent according to claim 9, wherein the garnet-structure is formed of M₃Fe₅O₁₂ and M=Y, La, Ce, Pr, Nd, Sm, Eu, Gd, Tb, Dy, Ho, Er, Tm, Yb, or Lu.

12. The contrast agent according to claim 9, wherein the magnetoplumbite-structure is formed of MFe₁₂O₁₉ and M=Ca, Sr, Ba, or Zn.

13. The contrast agent according to claim 9, wherein the hexagonal ferrite-structure is formed of Ba₂M₂Fe₁₂O₂₂ mit M=Mn, Fe, Co, Ni, Zn, or Mg.

14. The contrast agent according to claim 9, wherein the core (2) is additionally doped with Mn, Co, Ni, Cu, Zn, or F.

15. The contrast agent according to claim 14, wherein the amount of doping ranges between 0.01 and 5.00 mol-%.

16. The contrast agent according to claim 1, wherein the particle (1) further comprises at least one optional shell (3-5) on the core (2).

17. The contrast agent according to claim 16, wherein at least one of the optional shells (3-5) contains a radioactive isotope.

18. The contrast agent according to claim 17, wherein the radioactive isotope is ¹⁹F.

19. The contrast agent according to claim 17, wherein the radioactive isotope is present in an amount of 0.001 to 50 mol-%.

20. The contrast agent according to claim 17, wherein the at least one optional shell (3-5) containing the radioactive isotope has a thickness of 1 to 50 nm, preferably 1 to 10 nm.

21. The contrast agent according to claim 16, wherein the at least one optional shell (3-5) consists of precious metal, preferably Au, Pt, Ir, Os, Ag, Pd, Rh or Ru and more preferably Au.

22. The contrast agent according to claim 21, wherein the at least one optional shell (3-5) of precious metal covers the core (2) completely.

23. The contrast agent according to claim 21 wherein the at least one optional shell (3-5) of precious metal has a thickness of 1 to 50 nm, preferably 1 to 10 nm.

24. The contrast agent according to claim 16, wherein at least one further shell (3-5) is present, providing bio-compatibility.

25. The contrast agent according to claim 24, wherein the at least one biocompatibility shell (3-5) has a thickness of 1 to 50 nm, preferably 10 to 50 nm.

26. The contrast agent according to claim 16, wherein at least one further shell (3-5) is present, containing at least one antibody.

27. The contrast agent according to claim 26, wherein the at least one antibody is a tumor-specific antibody.

28. The contrast agent according to claim 26, wherein the at least one antibody containing shell (3-5) further contains one or more proteins, preferably the HIV-tat protein.

29. The contrast agent according to claim 1, wherein the core (2) has a spherical, oval or lens shape.

30. The contrast agent according to claim 1, wherein the core (2) has a diameter of 1 to 500 nm, preferably 5 to 50 nm.

31. A pharmaceutical formulation comprising a contrast agent and a pharmaceutically acceptable excipient, wherein the contrast agent is formed according to claim 1; and wherein the formulation is suitable for administration as an imaging enhancing agent and the contrast agent is present in an amount sufficient to enhance a magnetic resonance tomography (MRI) image, a magnetic particle imaging image, a positron emission tomography (PET) image, a single photon emission computed tomography (SPECT) image, a computed tomography (CT) image, or an ultrasound (US) image.

32. The pharmaceutical formulation of claim 31, wherein the pharmaceutical acceptable excipient is a buffered saline.
